(19) United States
(12) Patent Application Publication
(10) Pub. No.: US 2022/0044812 A1
(43) Pub. Date: Feb. 10, 2022

(54) AUTOMATED GENERATION OF STRUCTURED PATIENT DATA RECORD

(71) Applicant: Roche Molecular Systems, Inc., Pleasanton, CA (US)

(72) Inventors: Michael BARNES, Oro Valley, AZ (US); Anish KEJARIWAL, Pleasanton, CA (US); Weng Chi LOU, Pleasanton, CA (US); Margaret McCUSKER, Pleasanton, CA (US); Tyler J. O'NEILL, San Francisco, CA (US); Antoaneta VLADIMIROVA, Mountain View, CA (US); Yan XIAO, Pleasanton, CA (US); Stefanie BIENERT, Basel (CH); Matthew PRIME, Riehen (CH)

(21) Appl. No.: 17/445,475

(22) Filed: Aug. 19, 2021

Related U.S. Application Data
(63) Continuation of application No. PCT/US2020/019089, filed on Feb. 20, 2020.
(60) Provisional application No. 62/807,898, filed on Feb. 20, 2019.

(51) Int. Cl. G16H 50/20; G16H 10/60; G16H 50/70; G06F 40/40

(52) U.S. Cl. CPC G16H 50/20; G06F 40/40; G16H 50/70; G16H 10/60

(57) ABSTRACT

In one example, a method of extracting patient information for a medical application comprises: receiving patient data of a patient; processing the patient data using a learning system with Artificial Intelligence (AI)-assisted clinical extraction tool, the processing comprising: extracting, based on a trained language extraction model that reflects language semantics and a user's prior habit of entering other patient data, data elements from the patient data and data categories represented by the data elements, and mapping at least some of the extracted data elements to pre-determined data representations based on the data categories; populating fields of a data record of the patient based on the pre-determined data representations; and storing the populated data record in a database accessible by the medical application.

### CROSS REFERENCES TO RELATED APPLICATIONS

The present application is a continuation of International Patent Application No. PCT/US2020/019089, filed Feb. 20, 2020, which claims priority to U.S. Provisional Pat. Appl. No. 62/807,898, filed on Feb. 20, 2019, each of which is incorporated herein by reference in its entirety for all purposes.

### BACKGROUND

Every day, hospitals create a tremendous amount of clinical data across the globe. Analysis of this data is critical to understand detailed insights in healthcare delivery and quality of care, as well as provide a basis to improve personalized healthcare. Unfortunately, a large proportion of recorded data is difficult to access and analyze as most data are captured in an unstructured form. Unstructured data may include, for examples, healthcare provider notes, imaging or pathology reports, or any other data that are neither associated with a structured data model nor organized in a pre-defined manner to define the context and/or meaning of the data. Structured data may include data that are mapped to certain fields, codes, etc. that define the context and/or meaning of the mapped data, such that the meaning/context of the data can be determined based on the mapping.

Hospitals, as well as or other health care providers, try to address this limitation by using a combination of automated or semi-automated and manual processes as part of human-based abstraction to abstract unstructured data into structured data that can be readily interpreted based on the mapping. As part of an abstraction process, abstractors read various documents including unstructured data across a number of formats documenting the clinical encounter (typically electronic health records pathology reports, imaging reports, and laboratory reports), interpret these documents, and structure pertinent information into structured patient data records, such as a cancer registry. As used herein, a cancer registry can include an information system designed for the collection, management, and analysis of data on persons with the diagnosis of a malignant or neoplastic disease, such as cancer. The data stored in a cancer registry can be useful for many applications, such as performing quality of care analysis, cancer research, etc. But the process to manually extract and/or abstract such information into structured medical data records is laborious, slow, costly, and error-prone.

### BRIEF SUMMARY

Disclosed herein are techniques for a workflow to convert unstructured patient data into structured patients data records, such as a cancer registry, for a medical application. The medical application may include, for example, a quality of care evaluation tool to evaluate a quality of care administered to a patient, a medical research tool to determine a correlation between various information of the patient (e.g., demographic information) and tumor information (e.g., prognosis or expected survival) of the patient, etc. The techniques can also be applied to other registries, applications, etc. (e.g., an oncology workflow), and in other types of diseases areas.

In some embodiments, the techniques include receiving or retrieving patient data of a patient. The patient data can originate from various primary sources (at one or more healthcare institutions) including, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc. The patient data can include raw structured and unstructured patient data from the primary sources, as well as processed data (e.g. ingested, normalized, tagged, etc.) derived from the raw patient data.

The techniques may further include, as part of a workflow, processing the patient data using a learning system with an Artificial Intelligence (AI)-assisted clinical extraction tool. The learning system can include, for example, a rule-based extraction system, a machine learning (ML) model (which may include a deep learning neural network or other machine learning models), a natural language processor (NLP), etc., which can extract data elements from the unstructured patient data, classify (e.g., as part of a normalization process) the data elements, and map the data elements to pre-defined data representations (e.g., codes, fields, etc.) to form structured data based on the classification. A data representation may include data that is formatted/translated to a certain standard/protocol such that the data representation can be readily mapped to various data fields of a registry (e.g., a cancer registry). Moreover, as part of the normalization process, the learning system can also detect and correct data errors. The techniques can further include creating/updating a structured medical record, such as a cancer registry, based on the mapping of the data elements, and providing the structured medical record to a medical application for additional processing. The structured medical record can also be provided to other organizations to update other databases containing structured medical records, such as state cancer registries.

As part of the workflow, the AI-assisted clinical extraction tool can be continuously adapted based on new patient data. For example, some of the raw unstructured patient data from the primary sources can be post-processed (e.g., tagged) to indicate mappings of certain data elements as ground truth. The tagged unstructured patient data can be used to train the ML model and the NLP to perform the extraction, classification, and mapping. Moreover, rules of the rule-based extraction system can also be adapted based on the processed patient data to improve the error detection and correction processing. At least some of the tagging operations can be performed by abstractors to train the AI-assisted clinical extraction tool. The AI-assisted clinical extraction tool can then automatically perform the extraction, classification, mapping and correction on other patient data.

These and other embodiments of the invention are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

The detailed description is set forth with reference to the accompanying figures.

FIG. 1A and FIG. 1B illustrate an example of a structured patient data record and its potential applications.

FIG. 2 illustrates a system for converting unstructured patient data into a structured patient data record and providing data analytics on the structured patient data record, according to certain aspects of the present disclosure.

FIG. 3A, FIG. 3B, FIG. 3C, and FIG. 3D illustrate internal components and operations of the system of FIG. 2, according to certain aspects of the present disclosure.

FIG. 4A-FIG. 4G illustrate example display interfaces for interacting with the system of FIG. 2 to convert unstructured patient data into a structured patient data record, according to certain aspects of this disclosure.

FIG. 5, FIG. 6A, and FIG. 6B illustrate example display interfaces for interacting with the system of FIG. 2 to perform data analytics on the structured patient data record, according to certain aspects of this disclosure.

FIG. 7 illustrates a method of converting unstructured patient data into a structured patient data record, according to certain aspects of this disclosure.

FIG. 8 illustrates an example computer system that may be utilized to implement techniques disclosed herein.

### DETAILED DESCRIPTION

Disclosed herein are techniques for automated extraction of information into a structured patient data record, such as a cancer registry, based on learning system(s) with AI-assisted clinical abstraction and data normalization operations, and providing the structured patient data record to a medical application. The medical application may include, for example, a quality of care evaluation tool to evaluate a quality of care administered to a patient, a medical research tool to determine a correlation between various information of the patient (e.g., demographic information) and tumor information (e.g., prognosis results) of the patient, etc. The techniques can also be applied to other registries, applications, etc. (e.g., an oncology workflow), and in other types of diseases areas.

More specifically, patient data of a patient can be received or retrieved from multiple sources. The patient data can originate from various primary sources (at one or more healthcare institutions) including, for example, an EMR (electronic medical record) system, a PACS (picture archiving and communication system), a Digital Pathology (DP) system, a LIS (laboratory information system) including genomic data, RIS (radiology information system), patient reported outcomes, wearable and/or digital technologies, social media etc. The patient data can include raw structured and unstructured patient data from the primary sources, as well as processed data (e.g. ingested, normalized, tagged, etc.) derived from the raw patient data.

As part of a workflow, the patient data can be processed using a learning system with Artificial Intelligence (AI)-assisted clinical extraction tool. The learning system can include, for example, a rule-based extraction system, a machine learning (ML) model (which may include a deep learning neural network or other machine learning models), a natural language processor (NLP), etc., which can extract data elements from the unstructured patient data, classify the data elements, and map the data elements to pre-defined data representations (e.g., codes, fields, etc.) to form structured data. Data errors can also be detected and corrected. Examples of the unstructured patient data can include, for example, pathological report, doctor's notes, etc. The pre-defined data representations can include, for example, International Classification of Diseases (ICD), Systematized Nomenclature of Medicine (SNOMED), indications representing biographical information of the patient (e.g., identification, age, sex, etc.), indications representing medical history of the patient (e.g., tumor information, biomarker, history of treatments received, adverse events after the treatments, etc.), etc. Some of the received/retrieved patient data can also include structured data elements in these pre-defined data representations.

A structured patient data record can be updated/created based on the pre-defined presentations. For example, a cancer registry can include a structured data record of the patient including entries correspond to, for example, medical history of the patient, biographical information of the patient, etc. The pre-defined data representations (e.g., ontology representations such as ICD and SNOMED, biographical information, etc.) extracted and mapped from the unstructured patient data, as well as those obtained from the structured patient data, can be used to automatically populate corresponding entries of the data record in the cancer registry. In some embodiments, the pre-defined data representations can also be provided to an abstractor as suggestions to assist the abstractor in populating the entries of the data record.

Moreover, as part of the workflow, the AI-assisted clinical extraction tool can be continuously adapted to new patient data to improve the mapping and normalization processes. For example, some of the original unstructured patient data from the primary sources can be tagged to indicate mappings of certain data elements as ground truth. For example, a sequence of texts in doctor's notes can be tagged as a ground truth indication of an adverse effect of a treatment. The tagging can indicate, for example, a particular data category for a text string. The tagged doctor's notes can be used to train, for example, an NLP of the AI-assisted clinical extraction tool, to enable the NLP to extract text strings indicating adverse effects from other untagged doctor's notes. The NLP can also be trained with other training data sets including, for example, common data models, data dictionaries, hierarchical data (i.e. dependencies between/among text), to extract data elements based on a semantic and contextual understanding of the extracted data. For example, the natural language processor can be trained to select, from a set of standardized data candidates for a data element of the cancer registry, a candidate having a closest meaning as the extracted data. Moreover, some of the extracted data, such as numerical data, can also be updated or validated for consistency with one or more data normalization rules as part of the processing. Entries of the data records of the cancer registry can then be populated using the processed data.

The disclosed techniques can enable automated extraction of patient data from various sources, as well as conversion of the extracted patient data into structured patient data records, such as a cancer registry, which can substantially speed up the generation of structured patient data records. Moreover, using techniques such as natural language processing and data normalization, the likelihood of introducing data errors to the cancer registry can be reduced, which can improve the reliability of the abstraction extraction. Moreover, the cancer registry can include data elements to support clinical research and quality of care metrics computation. With the improvements in the overall speed of data flow and in the correctness and completeness of data and quality metrics, wider and faster access of high-quality patient data can be provided for clinical and research purposes, which can facilitate the development in treatments and medical technologies, as well as the improvement of the quality of care provided to the patients.

### I. Generating a Cancer Registry

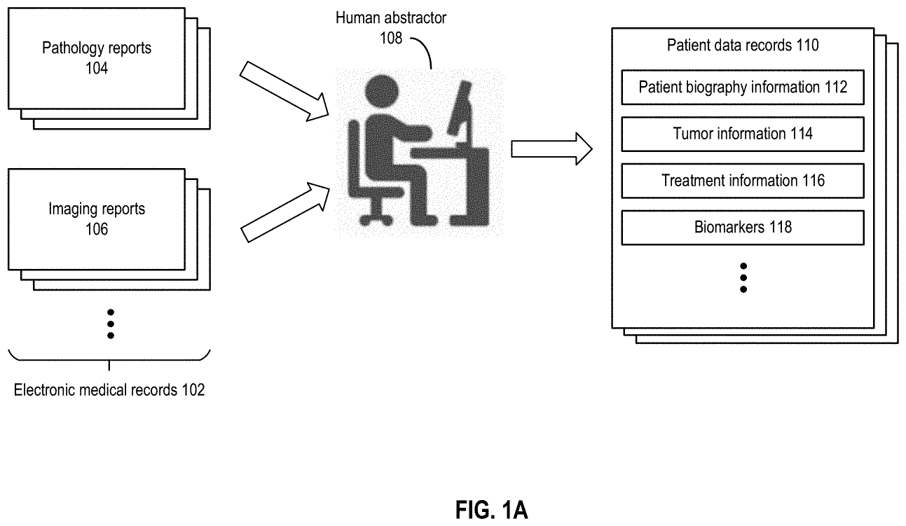

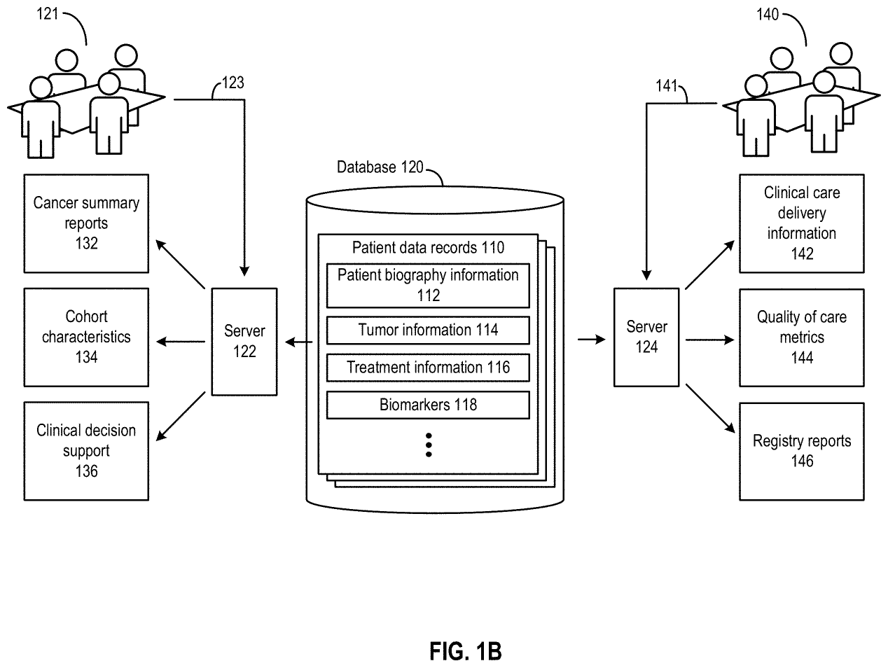

FIG. 1A illustrates a workflow for generating structured patient data records, such as a cancer registry, that may be improved by embodiments of the present disclosure. As shown in FIG. 1A, electronic medical records (EMR) 102 of a plurality of patients, such as pathology reports 104, imaging reports 106, etc., contain raw patients data. EMR 102 can be received and processed, in part, by a human abstractor 108 to populate data elements stored in patient data records 110 for a plurality of patients. Each patient data record 110 may include a plurality of sections or tables including a patient biography information section 112, a tumor information section 114, a treatment information section 116, a biomarkers section 118, etc. Each section can include multiple data elements (not shown in FIG. 1A). For example, patient biography information 112 may include data elements for names, demographic information, etc. Tumor information section 114 may include fields for procedure, specimen laterality, location, histologic type, etc. Human abstractor 108 can read and interpret medical data from electronic medical records 102, and populate the different data element fields of patient data records 110 for each patient with the medical data to convert the medical data into a structured form. The structured medical data of patient data records 110 can be provided to, for example, different medical applications including, for example, a clinical decision application, a care evaluation application, a research application, regional/national cancer registries, accreditation boards, etc. In some examples, patient data records 110 can include a cancer registry.

FIG. 1B shows patient data records 110 as part of an information system including a database 120 as well as servers 122 and 124 to provide access to the structured medical data for different medical applications and/or personnel. For example, servers 122 and 124 may include web servers to provide an interface for accessing database 120. As shown in FIG. 1B, epidemiologists/clinical researchers 121 can transmit a request 123 (e.g., a query) to server 122 to obtain structured medical data from patient data records 110 to generate cancer summary reports 132 (e.g., a report of patient population for each type of cancer, etc.) of all of the patients represented by patient data records 110 stored in database 120, cohort characteristics 134 (e.g., demographic characteristics of patients having the same type of tumor, etc.), clinical decision support 136 (e.g., to determine whether to administer a treatment based on treatment history and history of adverse effects from a pool patients), etc. The data used to generate cancer summary reports 132, cohort characteristics 134, and clinical decision support 136 may include data of, for example, patient information section 112, tumor information 114 section, treatment information 116, etc. of the cancer registry. As another example, hospital administrators and quality groups 140 can transmit a request 141 to server 124 to obtain structured patient data from database 120 to generate clinical care delivery information 142 (e.g., treatments administered by a caregiver), quality of care metrics 144 (e.g., to evaluate a quality of treatments/care administered by the caregiver), registry reports 146 to regional/national cancer registries, accreditation boards, etc. These data can be used to detect, for example, potential problems in the administration of care, and to find solutions to the problems. The data used to generate clinical care delivery information 142, quality of care metrics 144, registry reports 146 may come from, for example, tumor information section 114, biomarkers section 118, and treatment information section 116.

As discussed above, manual extraction of patient data from electronic medical records 102 (e.g., pathology reports, imaging reports, etc.) and conversion into patient data records can be a laborious, slow, costly, and error-prone process, which in turn affects performances and timeliness of the medical applications that rely on the cancer registry. For example, errors in the patient data records 110 can lead to generation of inaccurate cancer summary reports 132, cohort characteristics 134, clinical care delivery information 142, and quality of care metrics 144. Moreover, the slow and laborious data entry for patient data records 110 can also introduce delay in, for example, detection and remedy of problems in the administration of care.

### II. Automated Structured Medical Data Generation

The present disclosure proposes a data processing system that can perform automated extraction of patient data from electronic medical records and conversion into a structured patient data record, such as a cancer registry. The automated extraction can reduce or even eliminate the need for manual extraction and entry of patient data, which are slow and laborious as explained above. The data processing system can a learning such as, for example, a rule-based extraction system, a machine learning (ML) model (which may include a deep learning neural network or other machine learning models), a natural language processor (NLP), etc., to extract data elements from the unstructured patient data, classify the data elements, and map the data elements to pre-defined data representations (e.g., codes, fields, etc.) to form structured data, and then populate various fields of a structured patient data record (e.g., a cancer registry) based on the structured data. The data processing system can also operate in various modes, such as a full-automated mode in which the data processing system automatically populate the fields, or a hybrid mode in which some of the fields are populated by the data processing system while the rest of the fields are populated by a human abstractor. The hybrid mode can be part of the learning process to update the machine learning model.

A. System Overview

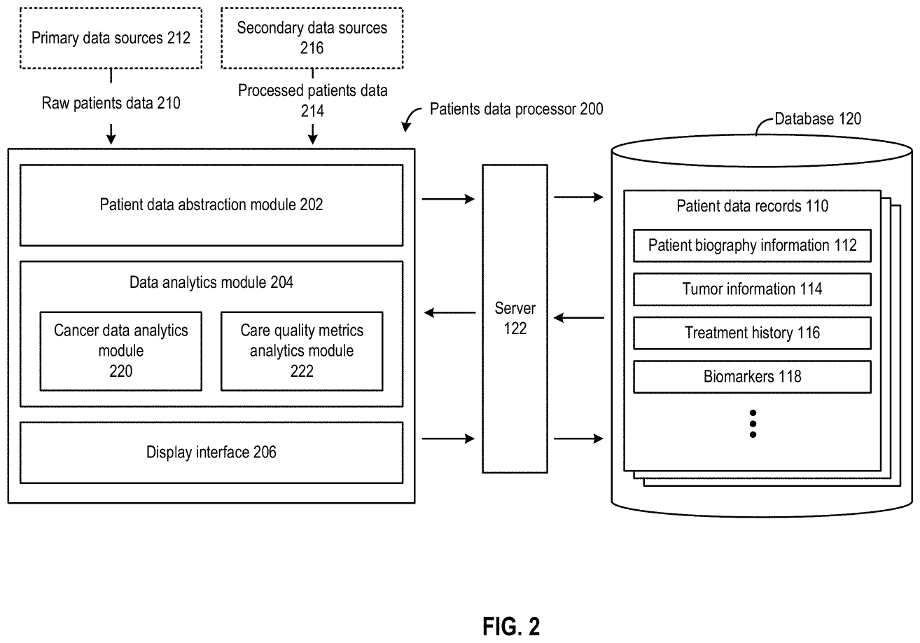

FIG. 2 illustrates an example patients data processor 200 according to embodiments of the present disclosure. As shown in FIG. 2, patients data processor 200 includes a patient data abstraction module 202, a data analytics module 204, and a display interface 206. In some examples, patient data processor 200 can be implemented in software and executed by one or more computer processors to implement the functions described below.

In some examples, patient data abstraction module 202 can receive raw patient data 210 of patients from primary data sources 212. Primary data sources 212 may include an EMR (electronic medical record) system, a PACS (picture archiving and communication system), a Digital Pathology (DP) system, an LIS (laboratory information system) including genomic data, an RIS (radiology information system), patient reported outcomes, wearable and/or digital technologies, social media, etc. Patient data processor 200 can perform an abstraction process of patients data, which include extraction of data elements from the raw patient data 210 and mapping the extracted data elements to various data element fields/entries of patient data records 110.

Patient data abstraction module 202 can perform abstraction of data using various techniques. For example, patient data abstraction module 202 can include a learning system with Artificial Intelligence (AI)-assisted clinical extraction tool. The learning system can include, for example, a rule-based extraction system, a machine learning (ML) model (which may include a deep learning neural network or other machine learning models), a natural language processor (NLP), etc., which can extract data elements from raw unstructured patient data (e.g., pathological report, doctor's notes, etc.), classify the data elements, and map the data elements to pre-defined data representations (e.g., codes, fields, etc.) to form structured data. The pre-defined data representations can include ontology representations including, for example, International Classification of Diseases (ICD) and Systematized. Nomenclature of Medicine (SNOMED). The data representations may also include indications representing biographical information of the patient (e.g., identification, age, sex, etc.), indications representing medical history of the patient (e.g., tumor information, biomarker, history of treatments received, adverse events after the treatments, etc.), etc. Moreover, the natural language processor can select, from a set of standardized data candidates for a data element field of the cancer registry, one or more candidates having the closest meaning as the extracted data.

Patient data abstraction module 202 can also perform data normalization on the numerical data (e.g., validating the expected range) to validate the numerical data, and to correct or flag invalid numerical data. The data normalization can be performed based on one or more data normalization rules. In some examples, raw patient data 210 may also include structured medical data having the pre-defined data representations, and patients data abstraction module 202 can extract data elements based on identifying the pre-defined presentations of the data elements.

Based on an operation mode, patient data abstraction module 202 can automatically populate different fields of patient data records 110 using the processed data, or assist an abstractor in populating the fields of patient data records 110. For example, in one operation mode, patient data abstraction module 202 can automatically populate, via server 122, different fields of patient data records 110 of database 120 based on pre-determined mapping between the pre-defined data representations and the fields of patient data records 110. Moreover, in a different operation mode, patient data abstraction module 202 may allow manual extraction as a backup option when, for example, AI-assisted clinical extraction tool outputs a low confidence level for the output, which may indicate that raw patients data 210 include data that are inconsistent with the training data set. In some examples, patient data abstraction module 202 may adopt a hybrid approach by allowing a human abstractor to populate certain data element fields, via a display interface 206 and server 122, while using the AI-assisted clinical extraction tool to populate other data element fields. Patient data abstraction module 202 may generate other information, such as a progress report for tracking the completion of a patient's data record, the percentages of fields being populated manually versus being populated automatically by the AI-assisted clinical extraction tool, etc., to facilitate the management of abstraction operations.

As part of the workflow, the AI-assisted clinical extraction tool can be continuously adapted, as described above. Specifically, patient data abstraction module 202 can receive processed patients data 214 from secondary data sources 216, such as a training data database, to train or adapt the models/rules for extracting data elements. Processed patients data 214 can be derived from some of the prior raw patients data 210 that have been processed (e.g., tagged) to indicate mappings of certain data elements as ground truth. The tagged raw patients data can be used to train the learning system (e.g., a ML model, an NLP, etc.) to perform the extraction, classification, and mapping processing. Moreover, rules of the rule-based extraction system can also be adapted based on the processed patient data to improve the error detection and correction processing. Processed patients data 214 can also be generated by the manual population of data element fields via display interface 206.

To further improve the quality of data stored in the patient data records 110 (e.g., the processed data reflecting the correct interpretation of the extracted data), the data of patient data records 110 can be validated as part of a periodic data curation process, which can be automated or handled manually on a regular basis. As part of the data curation process, any erroneous data in patient data records 110 can also be corrected. The learning system can be retrained based on the extracted data input and the desired processing output. Moreover, the one or more data normalization rules can be revised if incorrect normalization outputs are detected. As the learning system is re-trained using a more complete and accurate training data set, and the data normalization rules are also adjusted, the quality of processing output as well as the speed of processing can be improved.

After patient data abstraction module 202 populates patient data records 110 in database 120, data analytics module 204 can obtain data included in multiple sections of patient data records 110 from multiple patients included in database 120, and perform various analyses on patient data records 110. For example, in a case where patient data records 110 is part of a cancer registry, data analytics module 204 may include a cancer data analytics module 220 to perform analysis on data related to cancer types represented in patient data records 110 to generate, for example, cancer summary reports 132, cohort characteristics 134, etc. Moreover, a care quality metrics analytics module 222 can perform analysis on data related to a quality of care deliver to the patients represented in patient data records 110 to generate, for example, clinical care delivery information 142, quality of care metrics 144, etc. Further, patients data processor 200 may include a reporting module (not shown in FIG. 2) to transmit patient data records 110 to other entities, such as regional/national cancer registries, accreditation boards, etc.

Display interface 206 allows a user (e.g., an abstractor, an epidemiologist/clinical researcher, a hospital administrator, etc.) to interact with the patient data processor 200. For example, the display interface 206 allows the abstractor to instruct the patient data abstraction module 202 to perform automatic population of the fields of patient data records 110, to view the populated data, etc. Display interface 206 also allows a hospital administrator to retrieve and view reports of various quality of care metrics as well as other derived reports (e.g., accreditation report, etc.). The display interface 206 also allows a researcher to retrieve and view reports from cancer data analytics module 220 (e.g., cancer summary report, cohort characteristics, etc.). In some examples, as to be described below, the display interface 206 can be in the form of a dashboard which allows the user to select and customize the displayed information.

B. Patient Data Abstraction Module

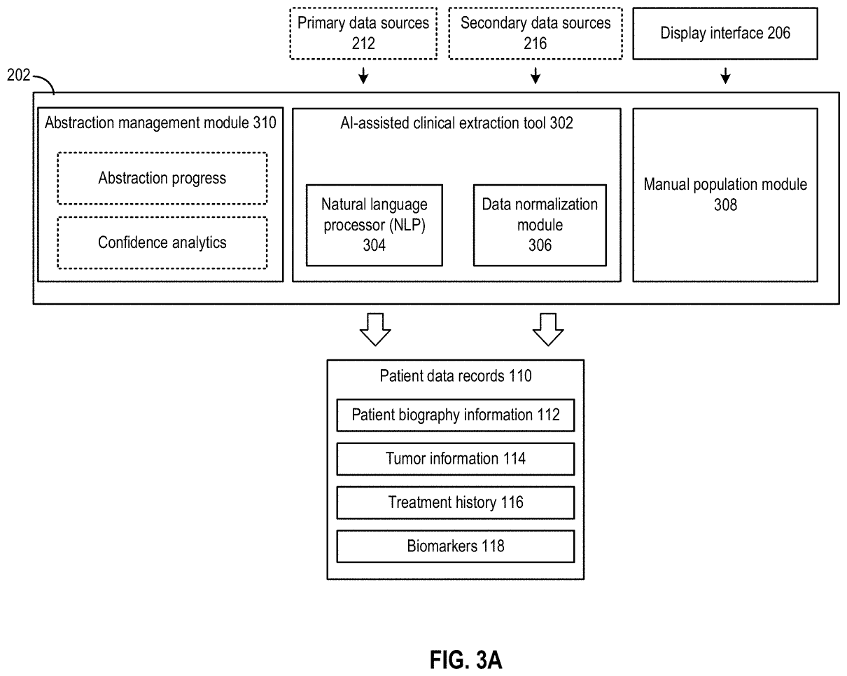

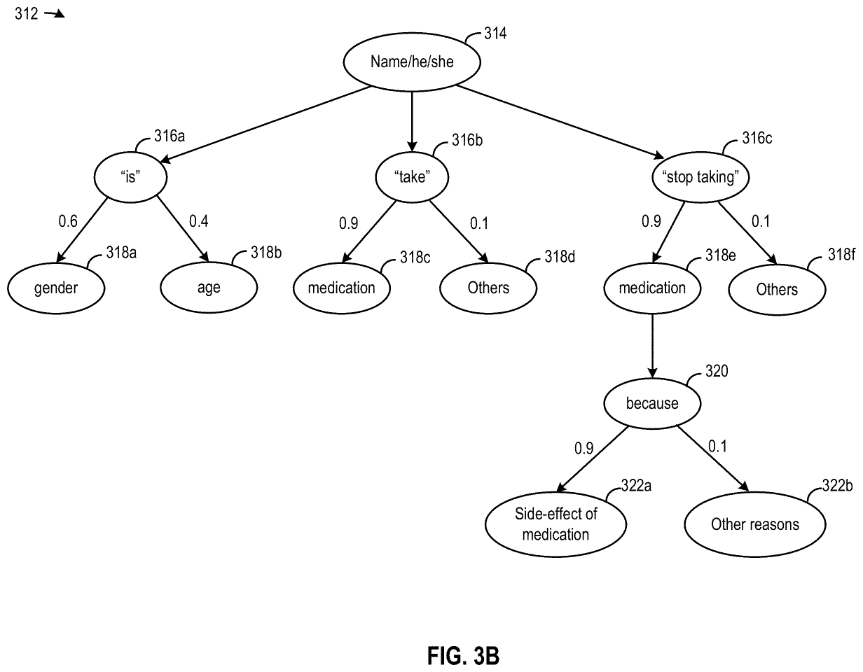

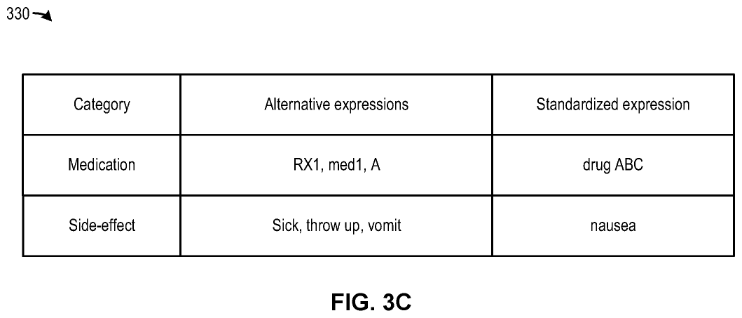

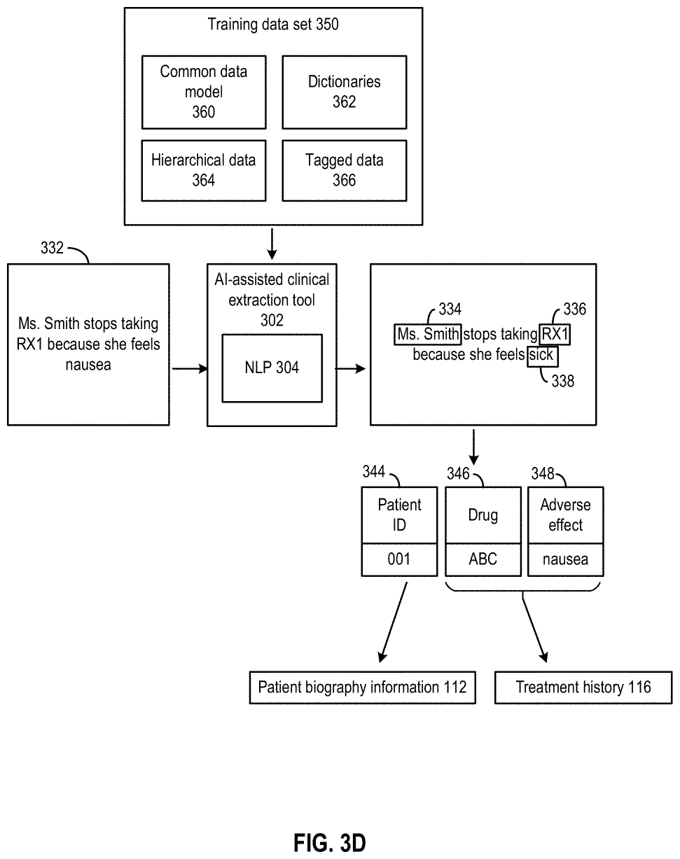

FIG. 3A illustrates an example of internal components of the patient data abstraction module 202, according to embodiments of the present disclosure. As shown in FIG. 3A, patients data abstraction module 202 includes an AI-assisted clinical extraction tool 302 which can include a learning system, such as a natural language processor 304, and a rule-based data normalization module 306, to perform extraction, mapping, and normalization of data elements from raw patients data 210, and to populate the corresponding entries of patient data records 110. Patients data abstraction module 202 also includes a manual population module 308 to enable manual population of the corresponding entries of patient data records 110. Patients data abstraction module 202 further includes an extraction analytics management 310 to manage various aspect of the extraction operations.

AI-assisted clinical extraction tool 302 can include a natural language processor 304 to extract data elements from unstructured raw patients 210, map the extracted data elements to a pre-determined data representation, and populate the fields of patient data records 110 that correspond to the pre-determined data representation.

FIG. 3B illustrates an example of a language extraction model 312 to support the extraction operations at natural language processor 304. As shown in FIG. 3B, language extraction model 312 can be in the form of a decision tree comprising nodes. Each node may represent a word/phrase identified from the raw data, or a predicted category/meaning of a subsequent word/phrase, while the nodes are connected by edges that connote a sequential relationship between two nodes and, in a case where the node represents a predicted category/meaning of a word/phrase, a probability that the prediction is accurate. The probability can reflect a user's habit of entering raw patients data 210 into primary data sources 212. As such, the decision tree can also reflect sequences of words/phrases according to semantics/structures of a sentence, as well as the user's habit.

Specifically, referring to FIG. 3B, node 314 of the decision tree can represent a name or a gender pronoun (he/she, etc.) of a patient subject. Node 314 is connected to nodes 316 including, for example, nodes 316a, 316b, and 316c, each representing a possible subsequent verb or word/phrase following the patient subject in a sentence. Each of nodes 316a, 316b, and 316c is also connected to nodes each representing a possible category/meaning of word/phrase that follows nodes 316a, 316b, and 316c. For example, node 316a is connected to node 318a representing gender and node 318b representing age, which represents that for a sequence of words/phrases represented by node 314 and 316a (e.g., “Jane Doe is”), the category of the word/phrase that follows can be a gender or an age of the patient subject. The probability of the following word/phrase belonging to a gender versus an age can be based on a user's habit as observed from other raw patients data 210 previously entered by the user and abstracted by patient data abstraction module 202. For example, based on the user's habit, there is a 60% chance (represented by “0.6” in FIG. 3B) that the word/phrase that follows “Jane Doe is” refers to a gender of the patient subject, while there is 40% chance (represented by “0.4” in FIG. 3B) that the word/phrase refers to an age of the patient subject. The probabilities can be based on the prior raw patients data entered by the user into primary data sources 212.

Moreover, node 316b is connected to a node 318c representing a medication category, as well as to a node 318d representing other categories. This represents that for a sequence of words/phrases represented by node 314 and 316b (e.g., “Jane Doe takes”), the category of the word/phrase that follows can be for a medication or other information, and there is a 90% chance (represented by “0.9” in FIG. 3B) that the word/phrase that follows refers to a medication. The probabilities can be based on the prior raw patients data entered by the user into primary data sources 212. The combination of nodes 314, 316b, and 318c can indicate that a patient subject takes a certain medication.

Further, node 316c is connected a node 318e representing a medication category with a 90% chance, as well as to a node 318f representing other categories. The combination of nodes 314, 316c, and 318e can indicate that a patient subject stops taking a certain medication. Node 318e is further connected to a set of nodes, including nodes 320, 322a, and 322b representing possible explanations of why the patient subject stops taking the medication. Node 322a represents a side-effect of the medication, whereas node 322b represents other reasons. There is a 90% chance that the phrase/word that follow node 318e refers to a side-effect of the medication, and there is a 10% chance that the phrase/words that follow node 318e refers to other reasons why the patient stops taking the medication. The probabilities can be based on the prior raw patients data entered by the user into primary data sources 212.

Natural language processor 304 can refer to the decision tree to determine a category of the word/phrase extracted from raw patients data 210. For example, if natural language processor 304 extracts a sequence of words/phrases “Jane Doe is”, which maps to a sequence of nodes 314 and 316a, natural language processor 304 can determine that the next word/phrase to be extracted more likely refers to a gender than an age of the patient. Also, if natural language processor 304 extracts a sequence of words/phrases “Jane Doe takes”, which maps to a sequence of nodes 314 and 316b, natural language processor 304 can that the next word/phrase to be extracted more likely refers to a medication taken by the patient. Further, if natural language processor 304 extracts a sequence of words/phrases “Jane Doe does not take”, natural language processor 304 can that the next word/phrase to be extracted more likely refers to a medication. If the sequence of nodes 314, 316b, and 318e is followed by words/phrases representing a reasoning statement (indicated by node 320), the reasoning statement is more likely to refer to a side-effect of the medication.

FIG. 3C illustrates a data table 330 to support the mapping and normalization of data elements by data normalization module 306. As shown in FIG. 3C, data table 330 can include map alternative expressions of a certain category, predicted based on language extraction model 312, to a standardized expression. For example, for a medication category, expressions such as “RX1”, “medl”, “A”, etc. can be mapped to the standardized expression “drug ABC”. Moreover, for a side-effect category, expressions such as “sick”, “throw up”, “vomit”, etc., can be mapped to the standardized expression “nausea”. Data table 330 can also reflect a user's habits of entering raw patients data 210 into primary data sources 212, such as the habits of using the short-handed expressions to represent certain information, and the mapping relationship in data table 330 can represent such habits.

While FIG. 3B and FIG. 3C illustrate that data categories for certain data elements are determined based on language extraction module 312 and then mapped to standardized expressions based on the data categories, it is understood that not all data elements need to be mapped based on their date categories. For example, a numerical value representing an age need not be mapped to standardized expressions. Rather, data normalization module 306 can compare the numerical value against a threshold range of age and determine whether the numerical value is valid, and correct the numerical value if it is outside the threshold range. The numerical value (corrected or not) can then be used to populate, for example, patient biography information 112 of patient data records 110.

FIG. 3D illustrates an example operation of a natural language processor (NLP) 304 and data normalization module 306. As shown in FIG. 3B, NLP 304 may receive text data 332. Text data 332 may include unstructured patients data and can be part of a doctor's note. NLP 304 can parse text data 332 and identify data elements 334, 336, and 338. NLP 304 can determine that data element 334 (“Ms. Smith”) corresponds to the name of a patient, data element 336 (“RX1”) likely corresponds to a medication/drug used by the author of the doctor's note, whereas data element 338 (“nausea”) likely corresponds to an adverse effect of a drug, based on language extraction model 312 of FIG. 3B.

Based on the determination of the categories of data elements 334, 336, and 338, data normalization module 306 can map each of data elements 334, 336, and 338 to, respectively, data representations 344, 346, and 348. For example, data representation 344 uses a patient identifier (“001”) to represent the patient's name (“Ms. Smith”). Data representation 346 uses a code (“ABC”), which can be based on SNOMED, ICD, or other standards, to represent the drug taken by Ms. Smith (“RX1”). Further, data representation 348 can link data element 338 (“nausea”) to a field representing the adverse effect developed by Ms. Smith as a result of taking drug ABC. At least some of the mapping can be based on data table 330 of FIG. 3C.

Each of data representations 344, 346, and 348 can correspond to various fields of a patient data record. For example, data representation 344 (patients identifier) can correspond to a patient's identifier field in patient biography information 112. Data representations 346 (drug) and 348 (adverse effect of the drug) can correspond to fields in treatment history 116 concerning a drug the patient has taken, and the adverse side effect the patient has developed from the drug. AI-assisted clinical extraction tool 302 can then populate the fields of patient data records 110 based on these data representations.

C. Training Operation to Perform Data Element Extraction

NLP 304 and data normalization module 306 (or other machine learning model, or a rule-based extractor) can be trained/adapted to identify data elements 334, 336, and 338 and their categories based on a training data set 350. Training data set 350 may include, for example, a common data model 360, dictionaries 362, hierarchical data 364, tagged data 366, etc., to identify data elements 334, 336, and 338 based on a semantic and contextual understanding of the extracted data developed through the training.

Specifically, a common data model 360 may define, for example, semantic structure of sentences, which enables NLP 304 to recognize a semantic structure and to deduce a meaning of a text based on the semantic structure and the text's location in the structure. Part of language extraction model 312 of FIG. 3B, such as the sequence of word/phrases represented by the nodes, can be built to reflect the semantic structure in common data model 360. Moreover, dictionaries 362 may provide, for example, translation between a foreign language and the English language, meanings of the texts or data elements, codes used by a particular doctor, etc. Dictionaries 362 may also provide standardization of the raw data. For example, “sex” may be reported in raw unstructured patients data as “male/female”, “m/f”, “0/1” and so forth. Dictionaries 362 may define a common data element structure such that, regardless of how the data are defined in the raw patients data, this data would be defined to a standardized format, e.g. “sex=0 (female), 1 (male), (missing)”, and the standardized data can be provided in a data representation and can be used to populate the corresponding fields of patient data records 110. Dictionaries 362 can be reflected in data table 330. Moreover, hierarchical data 364 may define certain dependencies between texts, which enables NLP 304 to extract a collection of texts that have meaning when put together. The sequence of text/phrases represented in language extraction model 312 of FIG. 3B can reflect hierarchical data 364.

In the example of FIG. 3B and FIG. 3D, based on common data model 360, dictionaries 362, and hierarchical data 364, language extraction model 312 can include a sequence of phrase/words representing a complete sentence starting with a subject followed by verbs, as well as the word “because” to define a reason. Based on language extraction model 312, NLP 304 may recognize “Ms. Smith” is a subject and is a name of a patient, whereas “stops taking RX1” is an action, whereas the word “because” defines that “nausea” is the reason for the action. NLP 304 may also recognize RX1 (e.g., from dictionaries 362) to represent the drug ABC, and “nausea” is a side effect. NLP 304 can then extract data elements 334, 336, and 338 based on such understanding and map the data elements to data representations 344, 346, and 348.

In addition, NLP 304 can also be trained by tagged data 366. Tagged data 366 may include raw unstructured patients data 210 which has been processed by, for example, having certain data elements tagged. The tagging can be performed by, for example, an abstractor, an administrator of patients data processor 200, etc. Tagged data 366 may include a similar pattern of data elements as text data 332, and the data elements can be tagged to indicate, for example, which data categories the data elements belong to, which data representations the data elements are mapped to as ground truth, etc. NLP 304 can be trained by tagged data 366 to, for example, update the probability of a word/phrase representing a certain data category in language extraction model 312. As a result, when NLP 304 receives untagged text data 332 including data elements 334, 336, and 338, NLP 304 can recognize the data pattern and determines the data representations for the data elements based on the recognized data pattern.

D. Data Normalization

Referring back to FIG. 3A, in addition to mapping the extracted data elements to standardized expressions based on data table 330, data normalization module 306 can also perform data normalization operations on extracted data. The data normalization operations can compare the extracted data targeted at a field against a reference range according to one or more data normalization rules, and adjust the extracted data based on a result of the comparison. The reference range may include, for example, a range of numerical values, a set of text, etc., which are considered as normal data for the field. For example, for extracted data targeted at a patient's weight field, data normalization module 306 can check the extracted weight value against a range of weights defined in the data normalization rules. If the extracted weight value exceeds the range of weights, data normalization module 306 can adjust the extracted weight value based on an error handling procedure defined in the data normalization rules. As an example, the error handling procedure may define that a number of rightmost zeros are to be removed from the extracted weight value such that the adjusted value falls within the range. As another example, data normalization module 306 can also perform standardization of the extracted data based on a data format/representation that is accepted by patient data records 110. For example, for a certain lab measurement, patient data records 110 may require the measurement to be listed as qualitative (e.g., positive/negative), whereas the extracted data is quantitative (e.g., having a numerical value), data normalization module 306 can compare the numerical measurement against a threshold to convert the numerical measurement to a qualitative representation acceptable by patient data records 110. The data normalization operations can also operate on unstructured text data by, for example, correcting a typo in the extracted text data by finding the closest text from a dictionary, etc.

In some examples, natural language processor 304 and data normalization module 306 can operate together in various ways to handle the extracted data. For example, the natural language processor 304 and data normalization module 306 can operate in parallel to handle different sets of extracted data. In one example, data normalization module 306 can be assigned to handle shorter text strings, numerical values, etc., for which data normalization rules can define a reference numerical range or a set of standardized text data candidates. Natural language processor 304 can be assigned to handle more complex text strings, which may require some forms of contextual and semantic analyses to determine the intended meaning of the text strings for the output. Data normalization module 306 and natural language processor 304 can also operate in a serial fashion on the same set of extracted data. For example, data normalization module 306 can perform pre-processing on the extracted data to correct typos and/or out-of-range values. Natural language processor 304 can then process the pre-processed data to generate an output associated with data elements in patient data records 110.

E. Manual Cancer Registry Population Assistance

Patient data abstraction module 202 further includes a manual population module 308, which allows a human abstractor to manually populate the fields of patient data records 110 via a display interface 206. The manual population module 308 can operate with AI-assisted clinical extraction tool 302 in various ways. For example, a display interface 206 can provide a selection option for each data element to select between automatic population and manual population. If automatic population is selected for a given data element, the AI-assisted clinical extraction tool 302 can extract the data from its primary data source(s) 212 tagged with a tag corresponding to the field, and populate the extracted data in the field. If manual population is selected, the user can enter the data for the field manually via the display interface 206. As another example, automatic population may be set as default, whereas manual population is provided as a backup when, for example, the confidence level of the natural language processor output is below a threshold.

F. Abstraction Management Module

Abstraction management module 310 can generate analytical results of the abstraction operations and manage the abstraction operations based on these results. For example, the extraction management module 310 can generate data-driven results reflecting the abstraction progress, such as percentage of completion of each patient's malignancy included in a given patient data record. The abstraction progress analysis results can also be aggregated at different levels, such as for different human abstractors assigned for the abstraction operations or for different caregivers (e.g., hospitals, clinics, etc.). The abstraction progress analysis results can be displayed via the display interface 206 and/or provided via other means to facilitate management of the abstraction operations. The abstraction progress analysis can also be used by abstraction management module 310 to track the progress of the automatic abstraction operations if the operations are fully automated. In addition, abstraction management module 310 can also generate results reflecting the confidence levels of the automatically populated data element fields (e.g., the confidence levels of the outputs of natural language processor 304). The confidence level can be based on, for example, a probability of a data element mapped to a particular data category as indicated in language extraction model 312. The confidence level information can be displayed via the display interface 206 to, for example, allow a user to select between automatic and manually populated data elements, as described above.

In addition, abstraction management module 310 can perform a routine cadence of data validation to improve the quality of data included patient data records 110 (e.g., the processed data reflecting the correct interpretation of the extracted data). The data curation process can be performed according to a management schedule. As part of the data curation process, the data of patient data records 110 can be validated and erroneous data can be corrected. Moreover, natural language processor 304 can be retrained based on the new extracted data and the one or more data normalization rules can also be revised if incorrect normalization outputs are detected. In some examples, the validation can be performed automatically by abstraction management module 310. For example, the natural language processor 304 can be retrained using a set of most recent extracted data. After the retraining, AI-assisted clinical extraction tool 302 can revisit earlier extracted data that have been processed and stored in patient data records 110, and reprocess those data with the retrained natural language processor 304. To further the data validation functionality and improve data quality included in patient data records 110, AI-assisted clinical extraction tool 302 can update the data of patient data records 110 if the data mismatch with the reprocessed data.

### III. Display Interface of Automated Structured Patient Data Generation

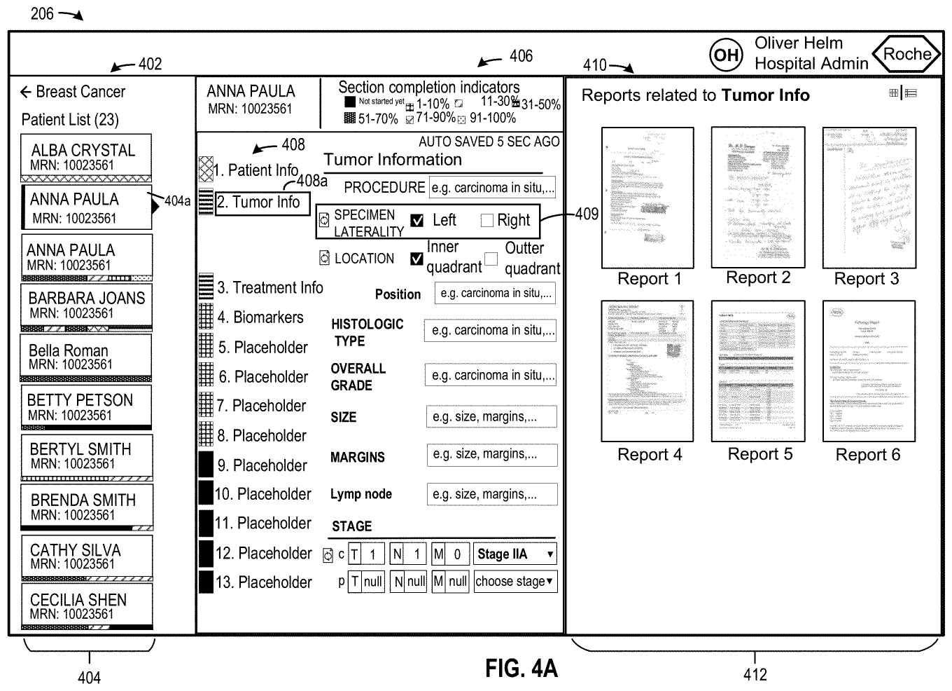

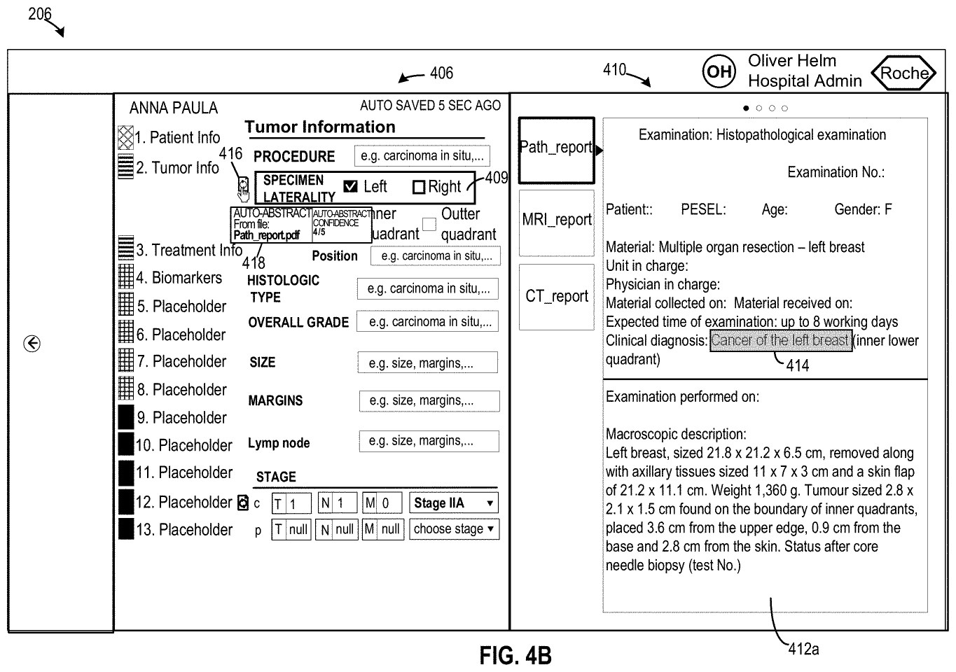

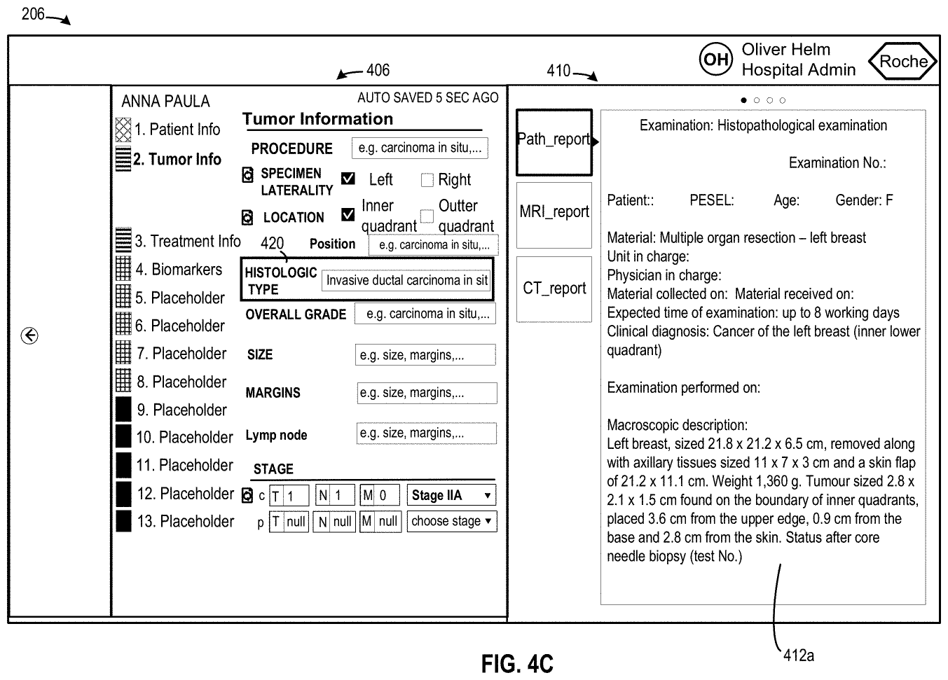

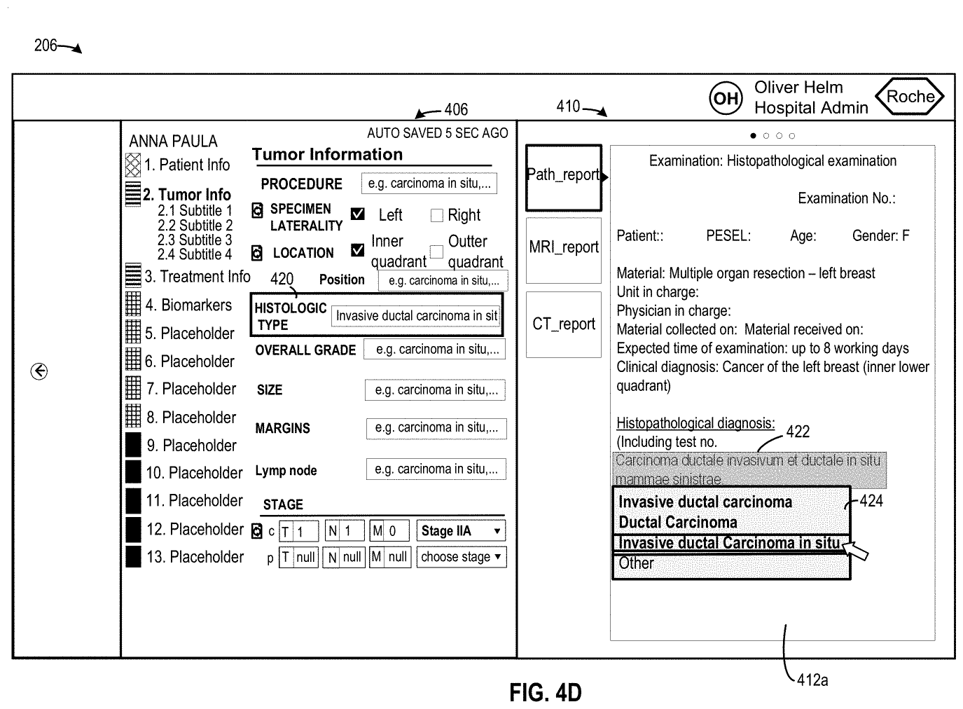

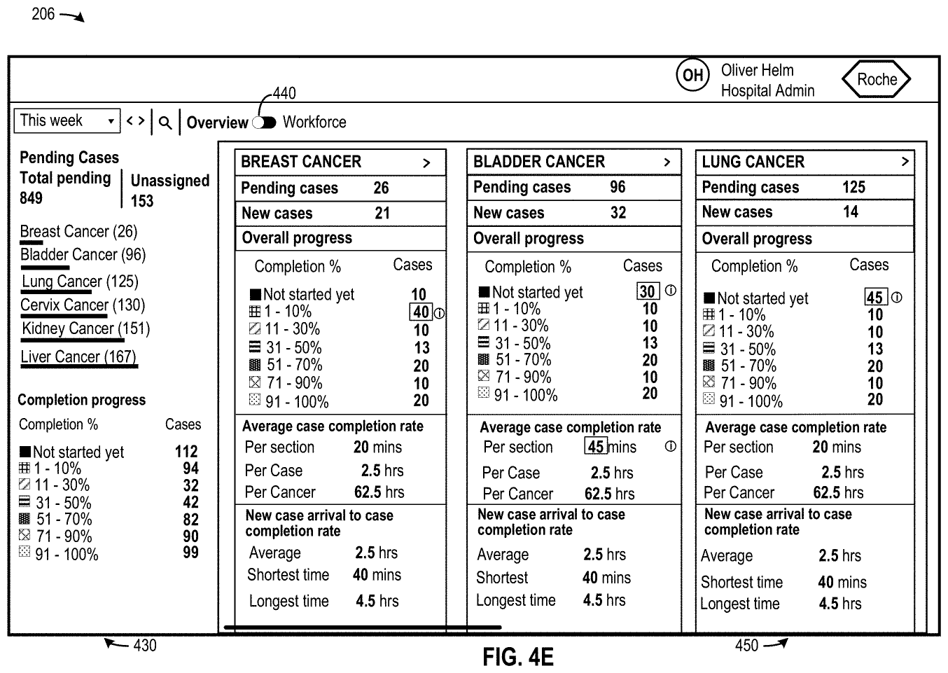

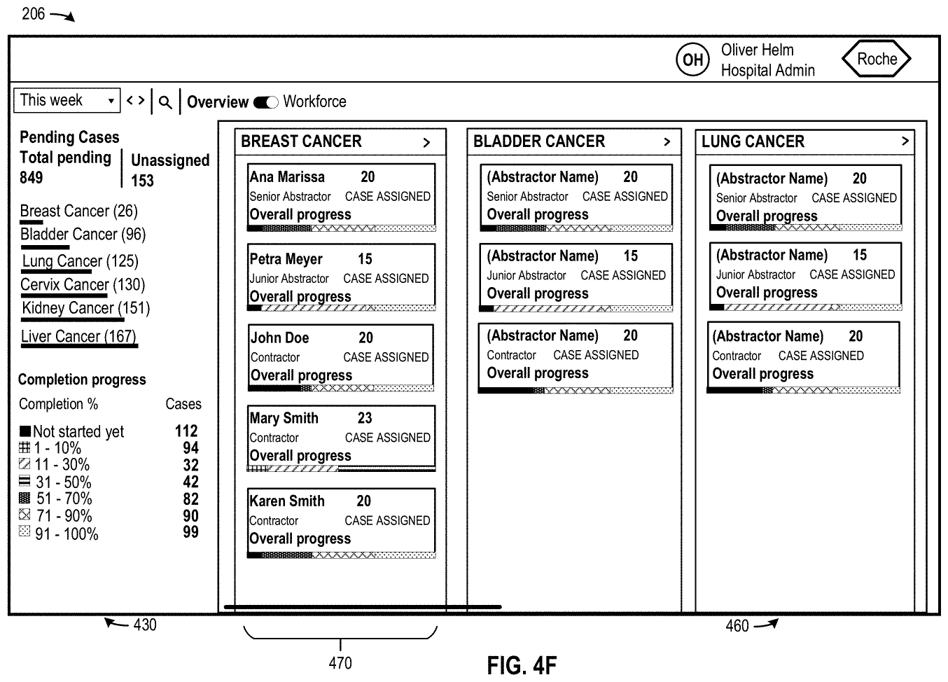

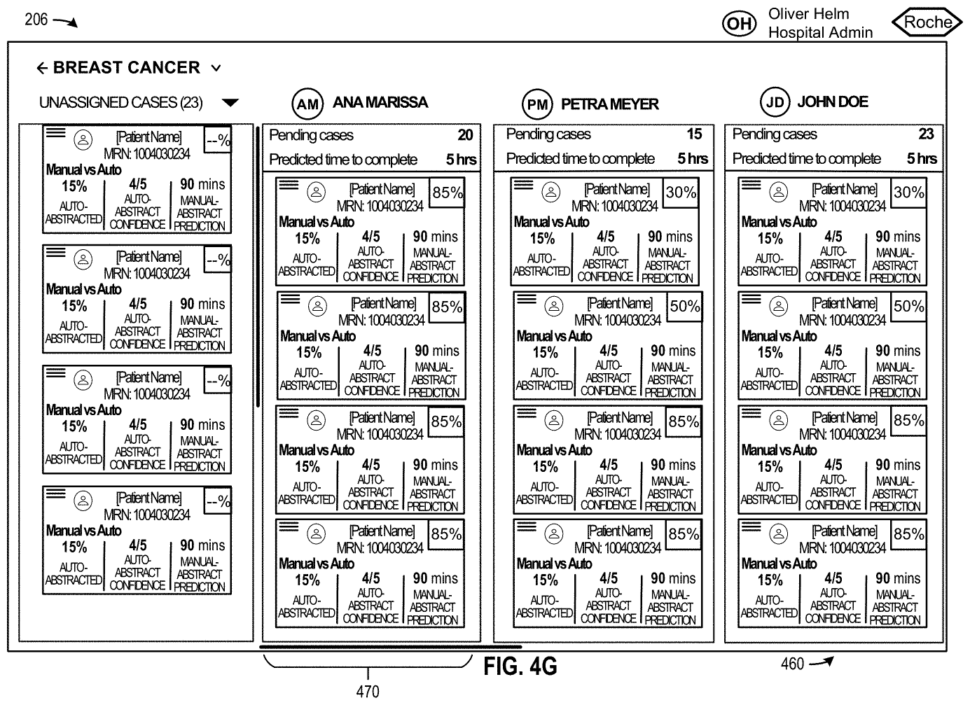

FIG. 4A to FIG. 4G illustrate examples of display interfaces 206 of patient data processor 200, according to embodiments of the present disclosure. As shown in FIG. 4A, the display interface 206 may include a patient section 402 (i.e. data table) that displays a list of selectable patient tabs 404, with each patient tab representing a single patient represented in patient data records 110. Selection of a patient tab (e.g., patient tab 404a) leads to displaying of a patient data record entry interface 406 for that patient. Patient data record entry interface 406 also displays a list of selectable section tabs 408, with each section tab representing a section of patient data records 110. For example, selection of the section tab 408a leads to displaying of the data elements and required fields of the tumor information section (e.g., 114 in FIG. 1) including field 409 (“Specimen laterality”). Display interface 206 further displays a document section 410. The document section 410 displays a set of thumbnails 412 each representing a document that provide the primary source of data to be extracted into the tumor information section 114. The documents can be obtained from a variety of external data sources 212. Some or all of the documents represented by thumbnails 412 may include raw patients data 210, as well as processed patients data 214 which may include tags.

FIG. 4B illustrates another view of the display interface 206 when a user selects field 409 displayed in patient data record entry interface 406. As shown in FIG. 4B, the selection of field 409 can cause document section 410 to expand one of the thumbnails 412, as illustrated in thumbnail 412a. The document section 410 can expand thumbnail 412a based on detecting that the document represented by thumbnail 412a contain processed patients data 214, which includes a tag 414 corresponding to field 409. Moreover, a selectable automatic population icon 416, as well as a pop-up message 418, are displayed adjacent to field 409. Upon selection, the automatic population icon 416 can cause AI-assisted clinical extraction tool 302 to extract the data tagged by tag 414 (e.g., by identifying the text or image of texts associated with tag 414), process the data using natural language processor 304, and populate field 409 with the processed data. The pop-up message 418 displays the name of the document file (“Path_report.pdf”) represented by thumbnail 412a, as well as a confidence level (4/5) of the processing by the natural language processor. As shown in FIG. 4B, based on processing, the extracted data tagged by tag 414 (“cancer of the left breast”), the option “left specimen laterality” is selected in field 409.

FIG. 4C and FIG. 4D illustrate other views of the display interface 206 when field 420 of tumor information section 114 (“histologic type”) is populated. As shown in FIG. 4C and 4D, the user can manually enter the data for a given data element field 420 via the display interface 206 or enable data for a given data element field be automatically populated. FIG. 4D shows that if text data tagged with a tag 422 correspond to data element 420 is detected, natural language processor 304 can process the text data to generate a number of standardized data candidates, which can be displayed in a pop-up window 424. A user can select one of the standardized data candidates and populate the data element field 420 with the selected candidate, as shown in FIG. 4D.

FIG. 4E-FIG. 4G illustrate other views of display interface 206 which display analytics on extracted data. Display interface 206 can provide a dashboard to display various types of information including, for example, a measurement of caseload to be extracted (e.g., the number of patients for whom a cancer registry is to be created), a measurement of caseload assigned to each abstractor, a progress report of creation of the cancer registries, assignment of the cases, etc. For example, as shown in FIG. 4E, display interface 206 can include a status summary 430 section that shows a total number of pending cases (e.g., patients for cancer registry creation) that are in progress, a total number of unassigned cases, a breakdown of the pending cases among different cancer types, a breakdown of the pending cases for different ranges of completion progress (e.g., measured by a percentage of completion), etc. In addition, the display interface 206 also provides a slide 440 for selecting a status display mode between an overview mode and a workforce mode. In a case where the overview mode is selected, the display interface 206 can display a detailed overview section 450 which provides additional progress metrics (e.g., case completion rates) for different cancer types.

FIG. 4F illustrates a detailed workforce section 460 displayed by a display interface 206 when the workforce mode is selected. As shown in FIG. 4F, the detailed workforce section 460 can display a set of abstractor tabs 470 for each cancer type, with each abstractor tab representing an individual abstractor assigned to extract the documents from various external sources into patient data records 110, such as a cancer registry, for a particular cancer type. Each abstractor tab is selectable. When selected, a detailed view of the progress metric for an abstractor can be displayed in detailed workforce section 460, as shown in FIG. 4G. As shown in FIG. 4G, the progress metrics for each abstractor may include, for example, a number of pending cases, the predicted time to complete, etc. The detailed workforce section 460 can also display the progress metrics of each pending case assigned to an abstractor. The progress metrics of each pending case displayed may include, for example, a percentage of fields populated by the AI-assisted clinical extraction tool 302, a confidence level of the output by the AI-assisted clinical extraction tool 302 for this case, a predicted time of completion if manual abstraction is performed, etc.

### IV. Automated Data Analysis Based on Structured Patient Data Records

Data contained with patient data records 110 can be procured by a data analytics module 204 to perform various automated analyses on the data. For example, as described above, cancer data analytics module 220 can generate, for example, cancer summary reports 132, describe cohort characteristics 134, etc. Moreover, care quality metrics analytics module 222 can generate, for example, clinical care delivery outcomes 142, quality of care metrics 144, etc. All these reports can also be displayed in an analytics dashboard provided by display interface 206. The analysis can be performed based on all or a subset of the patient data records 110 in database 120.

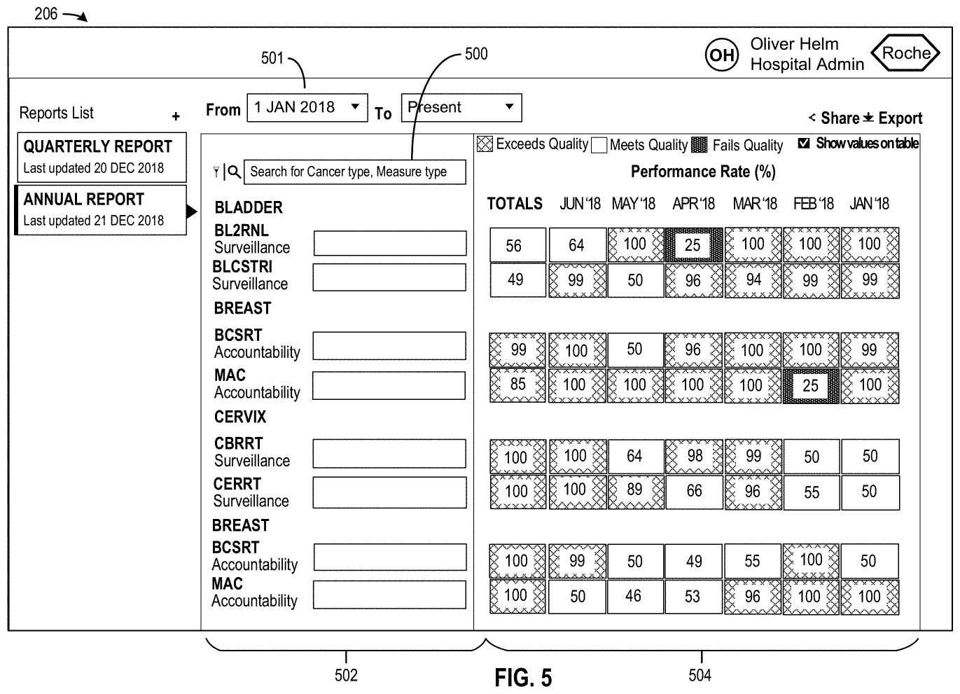

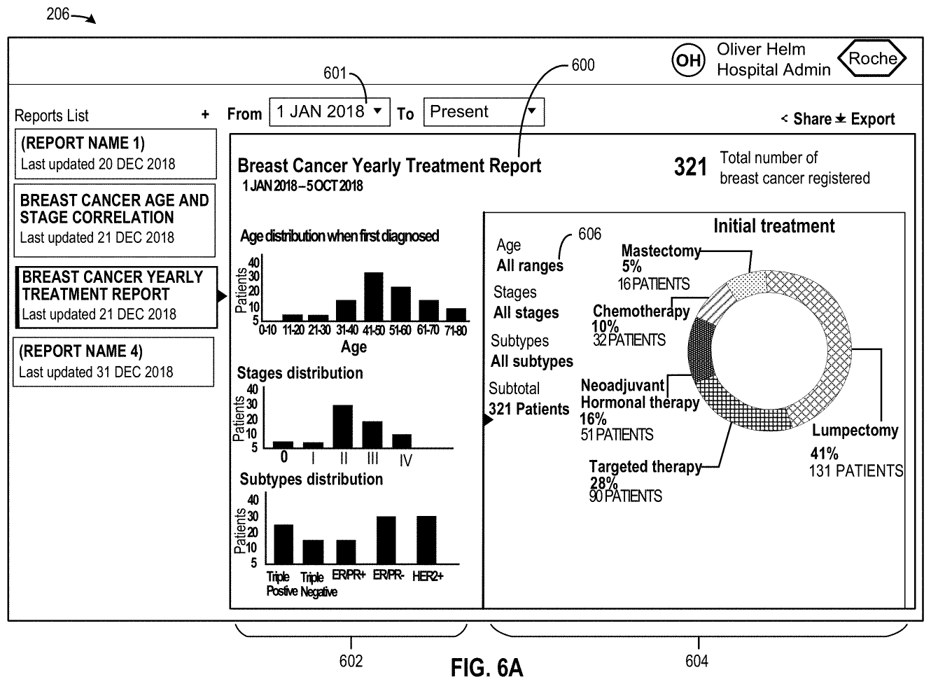

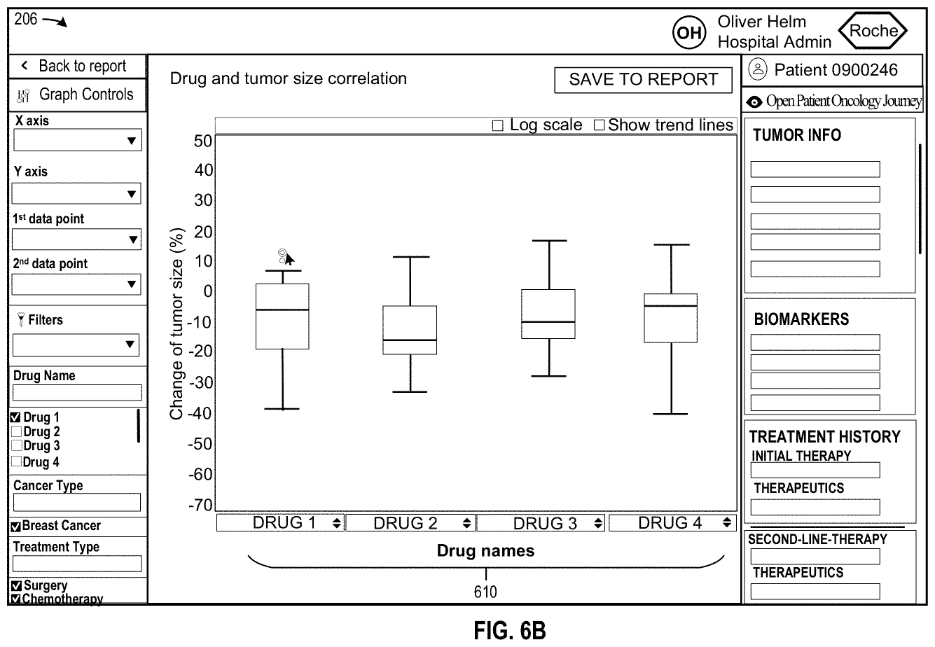

FIG. 5, FIG. 6A, and FIG. 6B illustrate examples of analytics dashboards provided by a display interface 206, according to embodiments of the present disclosure. As shown in FIG. 5, the display interface 206 may provide a care quality analytics dashboard 500 which displays performance measurements of a caregiver based on certain care quality metrics within a time period configured by the period selection boxes 501. For example, the care quality analytics dashboard 500 includes a care quality metrics section 502 which describes a set of care quality metrics (e.g., BL2RNL surveillance). Care quality analytics dashboard 500 further includes a performance rate section 504 that shows, for each care quality metric listed in the care quality metrics section 502, a percentage of new patients for whom the treatment satisfies the care quality metric and whether the percentage satisfies, exceeds, or fails a pre-defined threshold. The percentages can be categorized into different time periods to provide a distribution of the proportions stratified over time. The distribution allows a viewer (e.g., a caregiver management personnel) to identify time periods in which a substantial change in the proportions occurs, and the viewer can investigate the operations of the caregiver during that time period to identify potential causes of these changes.

Moreover, as shown in FIG. 6A, display interface 206 may provide a cancer analytics dashboard 600 which displays a breast cancer annual treatment report based on the data in patient data records 110. Based on the selected time period from the period selection boxes 601, patient information 112 (e.g., age), and tumor information 114 (e.g., stages and subtypes), the cancer data analytics module 220 can generate and display distribution graphs 604 based on age, stage, and cancer subtypes. Moreover, based on treatment history 116, the cancer data analytics module 220 can generate a distribution graph 604 displaying use of different treatments. The dashboard 600 further includes a configuration window 606 that allows a user to categorize patients (e.g., ages, cancer stages, cancer subtypes, etc.) represented in the distribution graphs 602 and 604. As another example, as shown in FIG. 6B, dashboard 600 can also display graphs 610 which shows data element central tendency and spread between the tumor size and different types of treatments, which the cancer data analytics module 220 can estimate based on the tumor information 114 and treatment history 116. The correlation graphs can be displayed for a single patient, as shown in FIG. 6B, or for a group of patients.

The analytics data shown in display interface 206 of FIG. 5, FIG. 6A, and FIG. 6B can become available as soon as the relevant and validated data are entered into patient data records 110. As a result, the timeliness of the results are of considerable value, and necessary to enact near real-time changes, versus the current approach to using data from cancer registries where such results are available typically on a quarterly or annual basis. Such arrangements allow the caregiver management to spot potential operation problems and cure the problems more quickly, which can improve the quality of care provided to the patients.

In addition, the patients data stored in patient data records 110 can be provided to different medical applications including, for example, a clinical decision application, regional/national cancer registries, accreditation boards, etc. For example, treatment history 116 can be used to predict the effect of treatment on a patient having similar characteristics (e.g., based on tumor information 114, biomarkers 118, etc.) as other patients whose records are stored in patient data records 110. Moreover, the patients data stored in patient data records 110 can be reported to regional/national cancer registries, accreditation boards, etc., to, for example, support affective oversight of the caregivers.

### V. Method

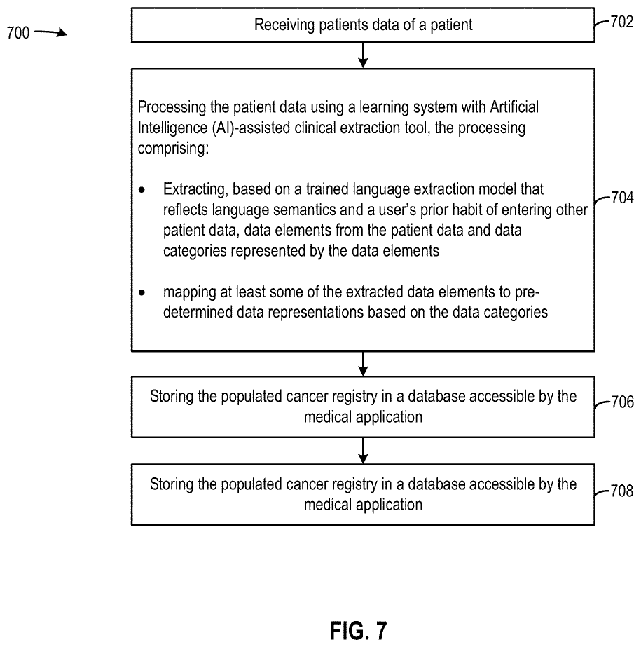

FIG. 7 illustrates a flowchart of a method 700 for abstracting patient data for a medical application, according to embodiments of the present disclosure. The method 700 can be performed by, for example, patients data processor 200 of FIG. 2.

In operation 702, the patient data processor 200 can receive patients data for an individual patient. The electronic medical records are received from one or more sources comprising at least one of: an EMR (electronic medical record) system, a PACS (picture archiving and communication system), a Digital Pathology (DP) system, an LIS (laboratory information system), a RIS (radiology information system), wearable and/or digital technologies, social media etc.

In operation 704, patient data processor 200 can process the patient data using a learning system with Artificial Intelligence (AI)-assisted clinical extraction tool (e.g., AI-assisted clinical extraction tool 302). The processing may include extracting, based on a trained language extraction model that reflects language semantics and a user's prior habit of entering other patient data, data elements from the patient data and data categories represented by the data elements, and mapping the extracted data elements to pre-determined data representations based on the data categories.

The learning system can include, for example, a rule-based extraction system, a machine learning (ML) model (which may include a deep learning neural network or other machine learning models), a natural language processor (NLP), etc., which can extract data elements from the unstructured patient data and determine their data categories based on a trained language extraction model, such as language extraction model 312 of FIG. 3B. Some of the data elements can also be mapped to pre-defined data representations (e.g., codes, fields, etc.) to form structured data, based on data table 330 of FIG. 3C. Moreover, as part of a normalization process, the learning system can also detect and correct data errors in the extracted data elements, and convert the extracted data elements to standardized data formats.

In operation 706, patient data processor 200 can populate fields of a data record of the patient corresponding to the data representations. The data representations (e.g., patients biography data, medication, side-effect, etc.) may correspond to certain fields of the data record, and the fields can be populated based on the corresponding data representations.

In operation 708, patient data processor 200 can store the populated patient data record in a database accessible by the medical application. The medical application may include, for example, a quality of care evaluation tool to evaluate the quality of care administered to a patient or patient population, a medical research tool to estimate a correlation between various information of the patient (e.g., demographic information) and tumor information (e.g., prognosis results) of the patient, a reporting tool to report the patient data record (e.g., a cancer registry) to a regional/national cancer registry, etc. The patients data processor 200 may include a data analytics module (e.g., data analytics module 204) to obtain data from sections (i.e. tables) included in the patient data record and to perform data analytics operations, with display of the data in a display interface (e.g., display interface 206), based on the techniques described above.

### VI. Computer System

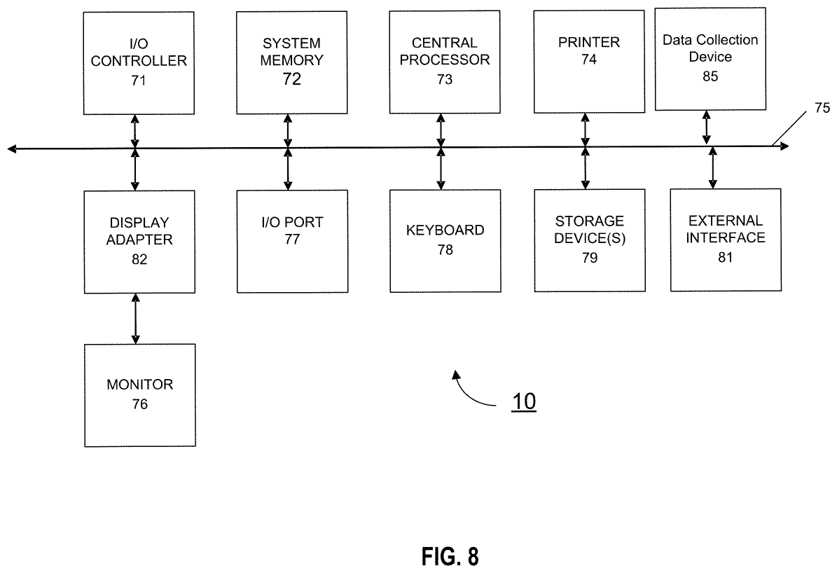

Any of the computer systems mentioned herein may utilize any suitable number of subsystems. Examples of such subsystems are shown in FIG. 8 in the computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 8 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect the computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A method of extracting patient information for a medical application, comprising:
receiving patient data of a patient;
processing the patient data using a learning system with Artificial Intelligence (AI)-assisted clinical extraction tool, the processing comprising:
extracting, based on a trained language extraction model that reflects language semantics and a user's prior habit of entering other patient data, data elements from the patient data and data categories represented by the data elements, and
mapping at least some of the extracted data elements to pre-determined data representations based on the data categories;
populating fields of a data record of the patient based on the pre-determined data representations; and
storing the populated data record in a database accessible by the medical application.

2. The method of claim 1, wherein the AI-assisted clinical extraction tool comprises a natural language processor;
wherein the language extraction model is trained using a set of training data comprising at least one of: a common text data model, dictionaries, hierarchical text data, or tagged text data;
wherein the language extraction model indicates probabilities of a data element representing multiple data categories, the probabilities being generated or updated by the training; and
wherein a data category associated with the highest probability is selected for the data element from the multiple data categories.

3. The method of claim 2, wherein the language extraction model is trained using the tagged text data, and wherein the tagged text data is derived from the other patient data and indicate at least one of: a data category for the text data, or a data representation mapped to the text data.

4. The method of claim 2, wherein the processing comprises converting the extracted data elements to a standardized data format based on a data table that maps multiple alternative expressions representing the same information to a single standardized expression.

5. The method of claim 2, wherein the processing comprises detecting an error in the extracted data elements based on comparing the extracted data elements against a threshold and updating the extracted data elements to remove the error;
and wherein the method further comprises populating the fields of the data record of the patient based on the updated extracted data elements.

6. The method of claim 1, further comprising:
displaying a first field in a user interface;
displaying, in the user interface, a first option to manually populate the first field of the data record and a second option to automatically populate the first field based on the data representations;
receiving, from the interface, a selection of the first option or the second option;
based on the selection, populating the first field with data received via a second field of the interface or with the data representations.

7. The method of claim 6, wherein the language extraction model indicates probabilities of a data element representing multiple data categories; and
wherein the method further comprises:
determining, based on probabilities indicated in the language extraction model, a confidence level of populating the first field based on the data representations; and
displaying the confidence level adjacent to the second option.

8. The method of claim 1, further comprising:
identifying a human abstractor responsible for abstracting patients data of a set of patients into data records of the set of patients;
determining a subset of the set of patients for whom the abstraction is incomplete;
determining a first percentage representing a ratio between the subset of the set of patients and the set of patients; and
displaying the first percentage and identification information of the abstractor in a second interface as part of a progress report of the abstractor.

9. The method of claim 8, further comprising:
determining a second percentage of completion of abstraction for the data record of each of the subset of the set of patients; and
displaying information related to the second percentages in the second interface as part of the progress report.

10. The method of claim 9, further comprising:
determining a predicted time of completion of manual population of remaining unpopulated fields of the data record of each of the subset of the set of patients; and
displaying the predicted time of completion as part of the progress report.

11. The method of claim 1, wherein the fields of the data record of the patient include tumor information and history of care;
wherein the medical application comprises a quality of care evaluation tool; and
wherein the populated data record enables the quality of care evaluation tool to determine a quality of care administered to the patient based on (1) the history of care and the tumor information included in the populated data record and (2) a quality of care metrics definition.

12. The method of claim 1, wherein the data elements of the data record of the patient include descriptive information of patients and tumor;
wherein the medical application comprises a medical research tool; and
wherein the populated data record enables the medical research tool to determine a correlation between descriptive information of the patients and descriptive information of the tumor included in the populated data record.

13. The method of claim 1, wherein the populated data record enables reporting to a regional and/or national data record of patients.

14. The method of claim 1, wherein the patients data are received from one or more sources comprising at least one of: an EMR (electronic medical record) system, a PACS (picture archiving and communication system), a Digital Pathology (DP) system, an LIS (laboratory information system), a RIS (radiology information system), patient reported outcomes, a wearable device, or a social media website.
